(19) United States
(12) Patent Application Publication
(10) Pub. No.: US 2024/0359260 A1
(43) Pub. Date: Oct. 31, 2024

(54) PROCESSING APPARATUS, METHOD FOR CONTROLLING THE PROCESSING APPARATUS, CONTROL DEVICE, COMPUTER PROGRAM AND COMPUTER-READABLE MEDIUM

(71) Applicant: SCHWIND EYE-TECH-SOLUTIONS GMBH, Kleinostheim (DE)

(72) Inventors: Samuel ARBA MOSQUERA, Aschaffenburg (DE); Shwetabh VERMA, Aschaffenburg (DE)

(21) Appl. No.: 18/645,792

(22) Filed: Apr. 25, 2024

(30) Foreign Application Priority Data
Apr. 28, 2023 (DE) 10 2023 111 156.6

(51) Int. Cl. B23K 26/0622; B23K 26/06; B23K 26/073

(52) U.S. Cl. CPC B23K 26/0622; B23K 26/0626; B23K 26/073

(57) ABSTRACT

The invention relates to a processing apparatus (1) for processing an object (9) by means of laser pulses (3), which at least comprises:
at least one laser beam source (2) configured for the emission of laser pulses (3), at least one beam exit device (4), which is configured to guide the respective laser pulses (3) with a predetermined laser pulse cross-sectional profile according to a pulse distribution profile (11) in a processing area (7) of a preset processing volume (8) of an object (9) to be processed, and a control device (10), which is configured to ascertain the pulse distribution profile (11) in the processing area (7) according to a predetermined pulse distribution ascertaining method for the preset processing volume (8) and the predetermined laser pulse cross-sectional profile. It is provided that the control device (10) is configured to transform the preset processing volume (8) into a deconvoluted processing volume (15) according to a predetermined deconvolution operation (F2) with the laser pulse cross-sectional profile as a point spread function of the deconvolution operation (F2), and to transform the deconvoluted processing volume (8) into the pulse distribution profile (11) by means of a predetermined conversion function (F5), in the predetermined pulse distribution ascertaining method.

### FIELD

The present invention relates to a processing apparatus for processing an object by means of laser pulses. In addition, the invention relates to a method for operating a processing apparatus for processing an object by means of laser pulses, to a control device for controlling a processing apparatus, to a computer program, and to a computer-readable medium.

### BACKGROUND

Processing apparatuses for laser-based processing a processing object are known from the prior art and are for example employed in the surface processing of processing objects of metal. A further field of application of the processing apparatuses for laser-based processing is the medical technology. In the medical technology, the laser-based processing apparatuses are for example applied for correcting an optical visual disorder and/or pathologically or unnaturally altered areas of the cornea. Therein, a pulsed laser and a beam focusing device can for example be formed such that laser pulses effect a photodisruption and/or photoablation in a focus situated within an organic tissue, to remove a tissue, in particular a tissue lenticule, from the cornea.

If the processing is an ablative removal of material of the object by means of laser pulses, it is often sought that a surface arising in the laser treatment has a surface roughness as low as possible. For example, this applies to a surface arising on the processing object, which is present after ablating a predetermined processing volume of the processing object. According to the current prior art, multiple approaches exist to reduce the surface roughness, which is caused by the ablative removal of material. For example, the approaches usual according to the prior art include optimization of a power distribution of a laser pulse.

U.S. Pat. No. 5,849,006 A discloses a system for laser reshaping the eye cornea. After calculating an ablation profile in accordance with the treatment of a certain eye complaint, a plurality of laser beam shots with uniform energy and fluence distribution is first selected to form a shot pattern with uniform shot density and thereby depth. Each shot removes a known amount of tissue volume, which is referred to as volume per shot (VPS) and ascertained by integration of the depth profile across the entire ablated region.

Despite of the mentioned optimizations, surfaces of a processing object further have a surface roughness too high for some specifications, after a certain processing volume has been removed by means of laser pulses.

### SUMMARY

Thus, it is an object of the invention to reduce a surface roughness arising in an ablation of a processing volume from an object by means of laser pulses.

This object is solved by the inventive processing apparatus according to the features of the embodiments presented herein, the inventive method according to the embodiments presented herein, the inventive control device according to the embodiments presented herein, the inventive computer program according to the embodiments presented herein and the inventive computer-readable medium according to the embodiments presented herein. Advantageous configurations with convenient developments of the invention are specified in the respective dependent claims, wherein advantageous configurations of each inventive aspect are to be regarded as advantageous configurations of the respectively other inventive aspects.

A first aspect of the invention relates to a processing apparatus for processing an object by means of laser pulses. In other words, the processing apparatus is provided, which is configured to process the object according to provided specifications to remove a processing volume from the object by means of laser pulses. The processing apparatus comprises at least one laser beam source, which is configured to emit the laser pulses required for processing the object. Preferably, the laser beam source can be an excimer laser, a gas laser, a solid-state laser, a laser diode, a fiber laser, preferably with a fiber oscillator and/or a fiber amplifier.

The processing apparatus comprises at least one beam exit device, which is configured to guide the respective laser pulses into a processing area of the preset processing volume of the object to be processed with a predetermined laser pulse cross-sectional profile according to a pulse distribution profile. In other words, the beam exit device is configured to guide the laser pulses emitted by laser beam source such that the laser pulses are guided onto preset positions of impingement of the object to be processed for processing the preset processing volume. The beam exit device is provided to guide the laser pulses onto the preset positions of impingement into the processing area on a surface of the object according to the pulse distribution profile.

The pulse distribution profile includes coordinates of the positions of impingement of the respective laser pulses to be adjusted on the object to be processed. Therein, the pulse distribution profile is related to the processing area, which is situated in the preset processing volume. In other words, the positions of impingement of the respective laser pulses are within the processing area. The processing volume is formed in a lateral plane of the surface of the object to be processed by the processing area and a processing depth. The processing depth can protrude into the object and relate to a depth, up to which material of the object is to be removed. The processing area and the processing volume can describe a contiguous area and a contiguous processing volume. The processing area can also include non-contiguous partial areas. Accordingly, the processing volume can include non-contiguous partial volumes. In other words, the processing area can include the partial areas spaced from each other. In this case, the partial volumes are also spaced from each other.

The laser pulses can comprise the predetermined laser pulse cross-sectional profile. The laser pulse cross-sectional profile can for example describe an intensity of the respective laser pulse depending on a distance to a center of the laser pulse in a cross-section of the laser pulse or a distribution of the intensity in a cross-sectional area of the respective one of the laser pulses. The laser pulse cross-sectional profile can also describe a distribution of the material ablation induced by the laser pulse. The intensity of the laser pulses can for example decrease from the center of the laser cross-section to an edge of the laser cross-section. The laser pulse cross-sectional profile can be caused by the processing apparatus, in particular by the laser beam source and the transfer optics, and be stored in the processing apparatus. It can also be provided that the laser pulse cross-sectional profile can be adapted in the processing apparatus by the control device or be selected from multiple possible laser pulse cross-sectional profiles. The adaptation of the laser pulse cross-sectional profile can for example be effected by a positioning or an opening of an aperture of the processing apparatus arranged in a beam path of the laser pulses.

It is provided that the at least one control device is configured to ascertain the pulse distribution profile in the processing area according to a predetermined pulse distribution ascertaining method for the preset processing volume and the predetermined laser pulse cross-sectional profile. In other words, the control device is provided to ascertain the coordinates of the positions of impingement of the respective laser pulses according to the pulse distribution profile in the processing area. Ascertaining the pulse distribution profile for the processing area by the control device is effected according to the predetermined pulse distribution ascertaining method for the preset processing volume. The control device is configured to ascertain the pulse distribution profile depending on the preset processing volume and the predetermined laser pulse cross-sectional profile of the laser pulses to allow processing of the processing volume of the object.

The control device is configured to adjust the ascertained pulse distribution profile in the at least one beam exit device. In other words, it is provided that the control device is configured to control the beam exit device such that the laser pulses are guided onto the respective positions of impingement in the processing area according to the pulse distribution profile.

It is provided that the control device is configured to transform the preset processing volume into a deconvoluted processing volume in the predetermined pulse distribution ascertaining method according to a predetermined deconvolution operation with the laser pulse cross-sectional profile as a point spread function of the deconvolution operation, and to transform the deconvoluted processing volume into the pulse distribution profile by means of a predetermined conversion function.

The pulse distribution ascertaining method includes an application of the predetermined deconvolution operation to the preset processing volume by the control device. The deconvolution operation is to be understood as a deconvolution operation in the mathematical sense and can for example also be referred to as deconvolution. The processing volume can be provided to the deconvolution operation as an area, wherein processing depths of the processing volume to be locally provided can be specified as a local intensity value of the area. The processing volume can for example be defined by means of a matrix, which can be defined in relation to the processing area. The matrix can include cells, which can be arranged in rows and columns. The rows and columns can be associated with respective directions in the processing area. The cells can be associated with respective positions or partial areas in the processing area and indicate respective processing depths, which the processing profile to be ablated is to have in the respective position. The point spread function can also be defined as a matrix with rows and columns, wherein cells of the matrix can describe respective intensities of the laser pulse cross-sectional profile in a cross-sectional area of the laser pulse. The cells can also describe respective processing depths in respective positions or partial areas of the cross-sectional area, which can describe a measure of an amount of material ablated by the respective laser pulse. For example, the matrix can describe the processing depth in Cartesian coordinates, in polar coordinates, in cylindrical coordinates or in spherical coordinates.

The pulse distribution profile can also be defined by means of a matrix, which can also be defined in relation to the processing area. The matrix can also include cells, which can be arranged in rows and columns. The rows and columns can also be associated with respective directions in the processing area. A number of the cells, the rows and the columns of the matrix of the pulse distribution profile can coincide with those of the matrix of the processing volume. The cells of the matrix of the pulse distribution profile can be associated with respective positions in the processing area, which can coincide with positions of impingement of the laser pulses. The cells can assign the respective number of laser pulses to the respective positions of impingement, which are to be guided onto the respective positions of impingement.

In order to ascertain the respective number of laser pulses, which are guided onto the respective positions of impingement of the laser pulses, for the preset processing volume, it is provided that the laser pulse cross-sectional profile is used as the point spread function in the deconvolution operation. Thus, the laser pulse cross-sectional profile can describe how an intensity distribution in the laser cross-section of a respective laser pulse is mapped to the surface of the object. The point spread function can for example also be defined such that it describes a local distribution of volume ablated on the object around a respective one of the positions of impingement or a local processing depth induced by the respective laser pulse. By performing the deconvolution operation on the processing volume with the laser pulse cross-sectional profile as the point spread function, the preset processing volume is transformed into a deconvoluted processing volume. The deconvoluted processing volume can also be defined as a matrix, wherein respective values of the cells of the matrix of the deconvoluted processing volume can have a quantitative magnitude arising based on the laser pulse cross-sectional profile. The quantitative magnitude can for example describe a theoretical local pulse number.

The at least one control device is configured to transform the deconvoluted processing volume into the pulse distribution profile by means of a predetermined conversion function. In other words, it is provided that the pulse distribution profile is generated based on the deconvoluted processing volume. The conversion function can for example include ascertaining the pulse number of a respective cell of the pulse distribution profile based on the value of the corresponding cell in the matrix of the deconvoluted processing volume. The case can also be present that the value of the matrix of the deconvoluted processing volume already describes the pulse number, which is transferred into the matrix of the pulse distribution profile by means of the conversion function in unchanged or adapted manner. The conversion function can also include an adaptation of a size of the matrix of the deconvoluted processing volume to the matrix of the pulse distribution profile or ascertaining the coordinates of the cells in the processing area.

The deconvolution is a mathematical operation, which generally outputs real numbers as values. The output values, which describe the number of the laser pulses to be output per position of impingement in this case, can thus be negative or no natural numbers. Negative values would correspond to providing material by the laser pulses, but which is not possible. Numbers, which are not natural numbers, would correspond to fractions of laser pulses. However, the laser beam source can possibly not be suitable for outputting laser pulse fractions, for example by means of a reduction of an energy of a laser pulse. For this reason, a conversion of the deconvoluted processing volume to the pulse distribution profile by means of the conversion function can be required.

The invention also includes developments, by which additional advantages arise.

In a development of the invention, it is provided that the control device is configured to adapt the preset processing volume according to a predetermined adaptation method depending on a pose of the processing area. In other words, it is provided that the preset processing volume is adapted to the adapted processing volume in the predetermined pulse distribution ascertaining method according to the predetermined adaptation method. In other words, it is provided that the control device is configured to apply the deconvolution operation not immediately to the preset processing volume, but to convert the processing volume into the adapted processing volume according to the predetermined adaptation method before the application of the deconvolution operation. The control device is configured to apply the deconvolution operation to the adapted processing volume, which has been generated from the preset processing volume in the predetermined adaptation method. The adaptation can for example be effected depending on a pose of the processing volume in the object and/or the pose of the processing volume in relation to the beam exit device. The adaptation can also depend on a structure of the material in the region of the processing volume and a geometry of the processing volume. The development results in the advantage that locally different material structures of the object and/or processing volumes of the laser pulses can for example be locally considered depending on a pose of the processing volume. For example, it can be provided that a processing volume of the laser pulses can depend on the pose of the respective position of impingement of the laser pulse in the treatment volume. An immediate application of the deconvolution operation to the preset treatment volume could result in processing of a treatment volume with certain geometries or dimensions of the treatment volume, which could too far deviate from the preset treatment volume. In other words, it can be relevant from which exit angle the laser pulses impinge on the object. This could result in the fact that more material is ablated by the respective laser pulses in one position of impingement than in another position of impingement. In the adapted processing volume, processing depths can be increased in predetermined positions, in which a material more difficult to ablate is for example located, with respect to the processing depths of the preset processing volume. By the artificial increase of the local processing depths, a lower local processing rate can be compensated for in a position such that the desired local processing depth is finally ablated. If the preset processing volume of the deconvolution operation would be supplied in ascertaining the pulse distribution profile for processing the material, this could result in the fact that corresponding local differences of the processing rates are not considered and the processing volume, which is actually ablated, deviates from the preset processing volume. By providing the adapted processing volume, locally caused deviations can be considered such that the ablated processing volume coincides with the preset processing volume despite of a deviation of the adapted processing volume from the preset processing volume.

A development of the invention provides that the control device is configured to perform the deconvolution operation in a frequency space. In other words, the control device is configured to transform the processing volume and the laser pulse cross-sectional profile into the frequency space. For example, it can be provided that the processing volume and the point spread function are transformed from the position space into the frequency space by a Fourier transformation. Thereby, the advantage arises that an efficient deconvolution of the treatment volume is allowed. A problem in performing the deconvolution operation in the frequency space is in that both the ablation volume and the laser pulse cross-sectional profile can include discrete values and can abruptly fall to zero outside of a certain range. This can result in so-called ringing effects upon performing the deconvolution operation in the frequency space. The control device can be configured to apply so-called padding methods for avoiding the ringing effects, wherein the processing volume is supplemented by additional cells in edge regions, which can form a frame around the processing volume. The inserted cells can for example have a padding value of 0.

A development of the invention provides that the control device is configured to perform the deconvolution operation in a position space. In other words, it is provided that both the processing volume and the point spread function are indicated in the position space and the deconvolution operation is performed in the position space. By the development, the advantage arises that ringing effects do not occur and thus an application of padding methods by the control device can be avoided.

A development of the invention provides that the control device is configured to perform a non-negative deconvolution as the deconvolution operation. In other words, it is provided that the deconvolution operation is defined such that negative values are not ascertained by the deconvolution operation. In other words, the deconvoluted processing volume is ascertained by the deconvolution operation, which does not have negative values. If the deconvoluted processing volume is described as a matrix, none of the cells has a negative value. By an application of a non-negative deconvolution as the deconvolution operation, the advantage arises that measures for avoiding non-physical pulse numbers can be avoided. Negative values of the deconvoluted processing volume would correspond to negative pulse numbers, which would mean adding material by laser pulses instead of ablating material by laser pulses. For resolving the negative circumstance, it would be required to ensure by further method steps that the pulse distribution profile does not have non-physical, negative values for the number of the pulses in the positions of impingement.

A development of the invention provides that the control device is configured to perform an integer deconvolution as the deconvolution operation. In other words, it is provided that the deconvolution operation is defined such that integers are ascertained as the values by the deconvolution operation. In other words, the deconvoluted processing volume is ascertained by the deconvolution operation, which has integers as the values. In particular, it can be provided that the integer deconvolution is a positive integer deconvolution, which is provided to output only positive integer values. Thus, the deconvolution operation is provided such that it only outputs integer values, in particular positive integer values. By the fact that only integer values are output, the advantage arises that it is not required to round the ascertained values of the deconvoluted processing volume for ascertaining the pulse number of the pulse distribution profile. Rounding to integer values could for example be required if the laser pulses can only be output with a single, not scalable intensity value. In particular, it is not required to compensate for the rounding deviations arising by the rounding by means of a post-hoc dithering method.

A development of the invention provides that the control device is configured to perform a binary deconvolution as the deconvolution operation. In other words, it is provided that the deconvolution operation is defined such that either the value 0 or the value 1 is ascertained by the deconvolution operation. The binary deconvolution can assign either the value 0 or the value 1 for the pulse number to the respective positions, onto which the pulses are guided. This can in particular be intended if the matrix defines a relatively high density of positions of impingement in the processing area.

A development of the invention provides that the control device is configured to round down the deconvoluted processing volume to a rounded deconvoluted processing volume by means of a rounding function. In other words, it is provided that the control device is configured to round real values of the pulses provided by the deconvolution operation, which can thus include fractions of pulses, to be able to obtain integers. For example, it can be provided that decimal places of the ascertained pulse number are rounded to be able to provide integer pulse numbers. Thereby, the advantage arises that a simple solution is provided to ascertain the pulse number for non-scalable laser pulses. For example, the rounding method can include rounding down the values of the cells of the deconvoluted processing volume to a smaller integer value. It can also be provided that a subtraction of a predetermined subtraction value from the pulse numbers is performed in addition to rounding down the pulse numbers. Therein, a preset boundary condition can be predetermined in order that the values of the rounded, deconvoluted processing volume remain positive despite of the subtraction.

A development of the invention provides that the control device is configured to assign a value of 0 to values of the deconvoluted processing volume with decimal places between 0 inclusive and 0.3 exclusive in the rounded down deconvoluted processing volume, to assign a laser pulse with a predetermined lower energy to values of the deconvoluted processing volume between 0.3 inclusive and 0.7 inclusive, and to assign an entire laser pulse to values of the deconvoluted processing volume from 0.7 exclusive in the rounded down deconvoluted processing volume, in the rounding function. In other words, it is provided that the rounding down function is configured to assign predetermined values to the pulse numbers of the deconvoluted processing volume depending on the decimal places of the pulse numbers, which can be associated with certain discrete energy values of the laser pulses. For example, it can be provided that it is rounded down to the lower integer with a decimal place below 0.3. In a range between 0.3 and 0.7, a predetermined pulse with a predetermined reduced pulse energy can be provided. From a decimal place of 0.7, the pulse number can be rounded up to the next integer value.

A development of the invention provides that the processing apparatus is formed to apply a post-hoc dithering method to the rounded deconvoluted processing volume depending on a deviation of the rounded deconvoluted processing volume from the deconvoluted processing volume to reduce the deviation. The application of the rounding function to the deconvoluted processing volume can result in the fact that the rounded deconvoluted processing volume has the deviation from the preset processing volume, which can be above a preset threshold value. The deviation arising by the rounding can be reduced by the application of the post-hoc dithering method. The post-hoc dithering method can include comparing the deconvoluted treatment volume, which includes the ascertained real values, to the rounded, deconvoluted treatment volume, in which the real values are rounded to the next non-negative integers. In the comparison, the deviation of the rounded deconvoluted treatment volume from the non-rounded deconvoluted treatment volume can be ascertained. The deviation ascertained via the deconvoluted treatment volume can be distributed over the rounded deconvoluted treatment volume in the post-hoc dithering method. In other words, the post-hoc dithering method is applied to the deviation between the volumes. The post-hoc dithering method assigns additional laser pulses to individual cells of the rounded down deconvoluted pulse distribution profile according to the dithering method, whereby the deviation caused by the rounding is reduced. The dithering method can include a suitable dithering method according to the prior art.

A development of the invention provides that the control device is configured to perform a predetermined refining method.

The control device is configured to convolute the rounded deconvoluted processing volume into a rounded convoluted processing volume by means of a convolution operation corresponding to the predetermined deconvolution operation with the laser pulse cross-sectional profile as a point spread function in the refining method. In other words, the control device is configured to perform a back-convolution, in which the rounded, deconvoluted processing volume is convoluted into the rounded convoluted processing volume, after rounding the deconvoluted processing volume to the rounded, deconvoluted processing volume. The rounded, convoluted processing volume describes a volume due to the rounding, which can deviate from the preset processing volume. The rounded, convoluted processing volume can be interpreted as that volume, which could arise in using the rounded, convoluted processing volume as a basis for determining the pulse distribution profile of the laser pulses of the point spread function.

The control device is configured to transform the rounded down convoluted processing volume according to a predetermined further deconvolution operation with a further laser pulse cross-sectional profile as a further point spread function of the further deconvolution operation. The further point spread function can differ from the point spread function. By performing the further deconvolution operation, a further deconvoluted processing volume is generated. In other words, the control device is configured to transform the convoluted, rounded down processing volume into the further deconvoluted processing volume according to the predetermined further deconvolution operation with the further laser pulse cross-sectional profile as the further point spread function of the further deconvolution operation and to transform the pulse distribution profile from the further deconvoluted processing volume.

Therein, the further laser pulse cross-sectional profile is used as the point spread function of the further deconvolution operation, wherein the further laser pulse cross-sectional profile of the further deconvolution operation can differ from the laser pulse cross-sectional profile used in the deconvolution operation. For example, it can be provided that the further laser pulse cross-sectional profile is assigned to a smaller energy than the laser pulse cross-sectional profile of the deconvolution operation. Thereby, the laser pulse cross-sectional profile can for example have lower heights and/or radii than the laser pulse cross- sectional profile of the deconvolution operation. The further laser pulse cross-sectional profile can in particular be smaller in its dimensions than the laser pulse cross-sectional profile and for example describe a further laser pulse, which has a smaller full width at half maximum than the laser pulse of the deconvolution operation. Therein, the further laser pulse cross-sectional profile can be preset by the structure of the processing apparatus or be adjusted by the processing apparatus according to specifications. For example, it can be provided that multiple laser pulse cross-sectional profiles are provided, from which one can be selected as the further laser pulse cross-sectional profile.

The development has the advantage that singularities can for example be avoided, which can occur upon abrupt changes between a transition zone and a functional optical zone or a treatment edge and a region of the cornea not to be treated.

A development of the invention provides that the control device is configured to perform a predetermined iteration method. The control device is configured to convolute the rounded deconvoluted processing volume into a rounded convoluted processing volume by means of a convolution operation corresponding to the predetermined deconvolution operation with the laser pulse cross-sectional profile as the point spread function in the iteration method and to define it as an initial volume of a first iteration step of an iteration method.

The control device is configured, in a respective step of the iteration method, to deconvolute the processing volume to a deconvoluted processing volume of the iteration step with a deconvolution operation of the iteration step with a laser pulse cross-sectional profile of the iteration step as a point spread function of the iteration step and to round the deconvoluted processing volume of the iteration step to a rounded deconvoluted processing volume according to a predetermined rounding method.

The control device is configured to convolute the rounded deconvoluted processing volume of the iteration step into a rounded convoluted processing volume of the iteration step by means of a convolution operation of the iteration step corresponding to the deconvolution operation of the iteration step with the laser pulse cross-sectional profile of the iteration step as a point spread function of the iteration step.

The control device is configured to ascertain a deviation between the rounded convoluted processing volume of the iteration step and the preset processing volume. The control device is configured to examine the deviation for satisfaction of a preset abortion criterion and to transform the rounded convoluted processing volume into the pulse distribution profile in case of satisfaction of the abortion criterion.

In case that the preset abortion criterion is not satisfied, the control device is configured to perform a further iteration step of the iteration method, wherein a laser pulse cross-sectional profile of the further iteration step as the point spread function of the further iteration step and/or the deconvolution operation as well as the convolution operation of the iteration step are adapted depending on the difference according to a predetermined specification. The iteration method can be performed until the abortion criterion is satisfied. For example, it can be provided that the predetermined specification provides a reduction of a radius of the laser pulse cross-sectional profile per iteration step. By the development, the advantage arises that a suitable laser pulse cross-sectional profile can be ascertained.

A development of the invention provides that the control device is configured to ascertain an initial matrix and to transform the initial matrix with a convolution operation with the point spread function into a convoluted initial matrix. The control device is configured to compare the convoluted initial matrix to the preset processing volume and to ascertain a deviation between the convoluted initial matrix and the preset processing volume. The control device is configured to examine the deviation for satisfaction of a preset abortion criterion and to transform the initial matrix into the pulse distribution profile by means of the conversion function in case of satisfaction of the abortion criterion. In case that the preset abortion criterion is not satisfied, the control device is configured to perform a further iteration step of the iteration method, wherein the initial matrix is adapted according to an adaptation function depending on the deviation according to a predetermined adaptation function.

The control device can be configured to scale at least some of the values of the initial matrix depending on local volume differences of the deviation by means of the adaptation function and to again transform the initial matrix, which has the scaled values, into the convoluted initial matrix by means of the convolution operation with the point spread function. The control device is configured to compare the further initial volume to the preset processing volume to ascertain the local volume differences between the convoluted initial matrix and the processing volume. The control device is configured to again scale at least one of the values of the initial matrix depending on the local volume differences. The control device is configured to repeat the steps and thus to perform an iterative adaptation of the initial matrix until a predetermined completion criterion is satisfied by the local volume differences. The control device is configured to define the initial matrix of the corresponding iteration step as the deconvoluted treatment volume upon satisfaction of the completion criterion and to transform it into the point distribution profile.

The control device can be configured to perform a direct convolution of the initial matrix, which can preset a pulse number for respective positions. The initial matrix can be initially filled with any initial values or initial values from a certain approximation method. For example, the initial matrix can have the initial value 1 in each position. The control device is configured to apply the convolution operation to the initial matrix with the laser pulse cross-sectional profile as the point spread function. The control device is configured to compare the result of the convolution operation, a resulting initial volume to the preset processing volume and to adapt the initial matrix depending on a local ratio or a local difference between the resulting processing volume and the preset processing volume. The adaptation can include upscaling or downscaling of at least one of the initial values of the initial matrix. The scaling can for example include an addition, subtraction, multiplication or division. This implementation is direct, but iterative. Herein, it is advantageous that the initial matrix natively has non-negative integers as values.

A second aspect of the invention relates to a method for operating a processing apparatus for processing an object by means of laser pulses.

In the method, it is provided that a pulse distribution profile in a processing area of the preset processing volume is ascertained for a preset processing volume according to a predetermined pulse distribution ascertaining method by at least one control device, which is configured for controlling the processing apparatus. The processing volume can describe a volume to be removed from the object by means of laser pulses, and be described by a preset pose on the object, preset dimensions and a preset geometry. The processing area can be an incision area of the processing volume with a surface of the object. The pulse distribution profile can describe positions of impingement of respective laser pulses in the processing area. The pulse distribution profile can be designed as a two-dimensional matrix, which can include rows and columns. A respective cell of the matrix can for example be associated with a respective position of impingement in the processing area, which can be arranged in a Cartesian pattern in the processing area. A respective one of the cells can describe a number of the laser pulses to be guided onto the respective position of impingement.

In the method, it is provided that the preset processing volume is transformed into a deconvoluted processing volume by the control device in the predetermined pulse distribution ascertaining method according to a predetermined deconvolution operation with the laser pulse cross-sectional profile as the point spread function of the deconvolution operation. The deconvoluted processing volume is transformed into the pulse distribution profile by means of a predetermined conversion function by the control device.

Subsequently, the ascertained pulse distribution profile is adjusted in the at least one beam exit device by the control device to guide respective laser pulses output by at least one beam exit device onto the positions of impingement in the processing area of the preset processing volume of the object to be processed according to the pulse distribution profile.

By the control device, control data for controlling the processing apparatus can be generated according to the method and sent to the processing apparatus.

The control data can include a respective dataset for positioning and/or for focusing individual laser pulses on a surface of the object. Additionally or alternatively, a respective dataset for adjusting at least one beam device for beam guidance and/or beam shaping and/or beam deflection and/or beam focusing of a laser beam of the respective laser can be included in the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A third aspect of the invention relates to a control device, which is formed to perform the steps of at least one embodiment of one of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A fourth aspect of the invention relates to a computer program. The computer program includes commands, which for example form a program code. The program code can include at least one control dataset with the respective control data for the respective laser. Upon execution of the program code by means of a control device, which can for example be configured as a computer or a computer cluster, it is caused to execute the previously described method or at least one embodiment thereof.

A fifth aspect of the invention relates to a computer-readable medium, on which the above mentioned computer program and the commands thereof, respectively, are stored. For executing the computer program, a control device, which can for example be designed as a computer or a computer cluster, can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figures. The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a processing apparatus, according to an embodiment.

FIG. 2 depicts a schematic representation of a deconvolution operation, according to an embodiment.

FIG. 3 depicts a schematic representation of a sequence of ascertaining a pulse distribution profile according to an iterative method, according to an embodiment.

FIG. 4 depicts a further schematic representation of a sequence of ascertaining a pulse distribution profile according to an iterative method, according to an embodiment.

FIG. 5 depicts a schematic representation of a sequence of ascertaining a pulse distribution profile according to a further iterative method, according to an embodiment.

FIG. 6 depicts a schematic representation of a sequence of a further method for operating a processing apparatus, according to an embodiment.

### DETAILED DESCRIPTION

In the figures, identical or functionally identical elements are provided with the same reference characters.

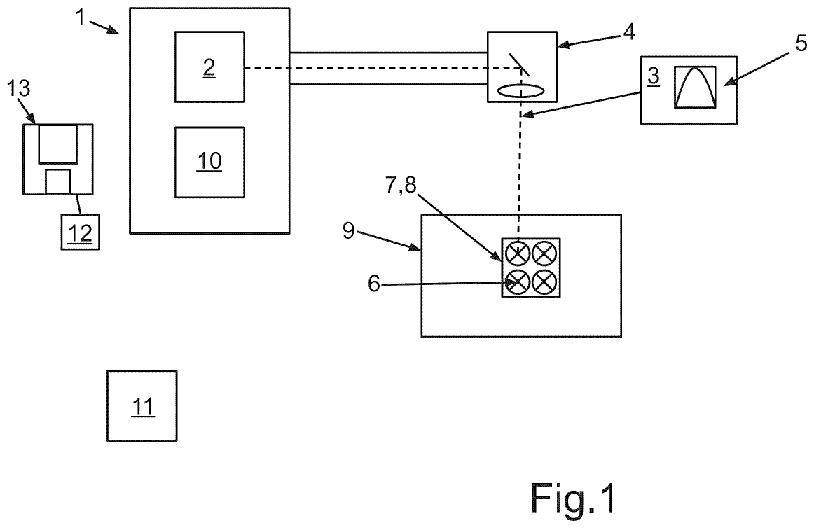

FIG. 1 shows a schematic representation of a processing apparatus.

The processing apparatus 1 comprises at least one laser beam source 2, which can be configured to emit laser pulses 3. The processing apparatus 1 can additionally comprise at least one beam exit device 4, which can be configured to guide the respective laser pulses 3 to respective positions of impingement 6 onto a processing area 7 of a preset processing volume 8 of an object 9 to be processed. The respective laser pulses 3 can have a predetermined laser pulse cross-sectional profile 5, which can be preset by the processing apparatus 1 or can be adjusted at the processing apparatus 1. The processing apparatus 1 can comprise a control device 10, which can be configured to control the beam exit device 4 and the laser beam source 2 for ablating the processing volume 8. The control device 10 can be configured to adjust the laser pulse cross-sectional profile 5 or to select it from a selection of possible laser pulse cross-sectional profiles 5.

For ablating the processing volume 8, it can be provided that the beam exit device 4 is controlled by the control device 10 such that the laser pulses 3 are guided onto predetermined positions of impingement 6, which can be preset by a pulse distribution profile 11. For ascertaining the pulse distribution profile 11, it can be provided that the control device 10 is configured to perform a predetermined pulse distribution ascertaining method to ascertain the pulse distribution profile 11 for the preset processing volume 8. Therein, the pulse distribution profile 11 can be defined such that upon supply of the laser pulses 3 to the respective positions of impingement 6 of the pulse distribution profile 11, processing of the preset processing volume 8 from the object 9 can be effected. The pulse distribution profile 11 can be ascertained from the preset processing volume 8 according to the pulse distribution ascertaining method, wherein the preset processing volume 8 is transformed into a deconvoluted processing volume 15 according to a predetermined deconvolution operation with the laser pulse cross-sectional profile 5 as a point spread function by the control device 10. The preset processing volume 8 can for example describe a three-dimensional body and preset a processing area 7, which can have respective values of a processing depth of the processing volume 8 in respective positions. The laser pulse cross-sectional profile 5 of the respective laser pulse 3 can be used as the point spread function, wherein the laser pulse cross-sectional profile 5 can for example describe a power density or an ascertained material ablation by the respective laser pulse 3 in a two-dimensional area.

The laser pulse cross-sectional profile 5 can be preset by the processing apparatus 1 and be unchangeable, wherein the laser pulse cross-sectional profile 5 can be stored in the control device 10. Alternatively, the laser pulse cross-sectional profile 5 can be adjusted, selected and/or adapted at least to a certain extent by the control device 10. The deconvoluted processing volume 8 can be transformed into the pulse distribution profile 11 by means of a predetermined conversion function by the control device 10. According to the ascertained pulse distribution profile 11, the control device 10 can control the laser beam source 2 and/or the beam exit device 4 to supply the individual laser pulses 3 to the respective positions of impingement 6 as they are preset by the pulse distribution profile 11. It can be provided that a processing efficiency of the laser pulses 3 can vary across the processing area 7. The processing efficiency can for example depend on an angle of impingement of the laser pulse 3 on the object 9 or on local material deviations of the object 9. Thereby, the material ablation by a respective one of the laser pulses 3 can vary depending on the pose of the position of impingement 6. In order to be able to consider the deviation of the processing efficiency, it can be provided that the control device 10 is configured to perform a predetermined adaptation method to change the preset processing volume 8 into an adapted processing volume 14. Therein, the deconvolution operation and the subsequent ascertainment of the pulse distribution profile 11 can be performed based on the adapted processing volume 8. The adapted processing volume 8 can for example differ from the preset processing volume 8 in a distribution of the processing depth depending on the lateral pose of a point. For example, it can be provided that a depth of the adapted processing volume 8 is increased with respect to the preset processing volume 8 to be able to compensate for a lower ablation efficiency of a laser pulse 3. The deconvolution operation can be a preset deconvolution operation according to the prior art, which can for example be applied to the processing volume 8 in a position space or a frequency space by the control device 10. The deconvolution operation can for example be a non-negative deconvolution. It can be provided that the control device 10 is configured to perform a predetermined rounding method to round the pulse numbers ascertained for respective positions of impingement 6 to integer values. It can also be provided that a laser pulse 3 with a reduced energy can be associated with certain decimal places instead of a certain, entire laser pulse 3.

In order to be able to allow the operation to the control device 10, it can be provided that a computer program 12 is provided to the control device 10, which can be stored on a computer-readable medium 13.

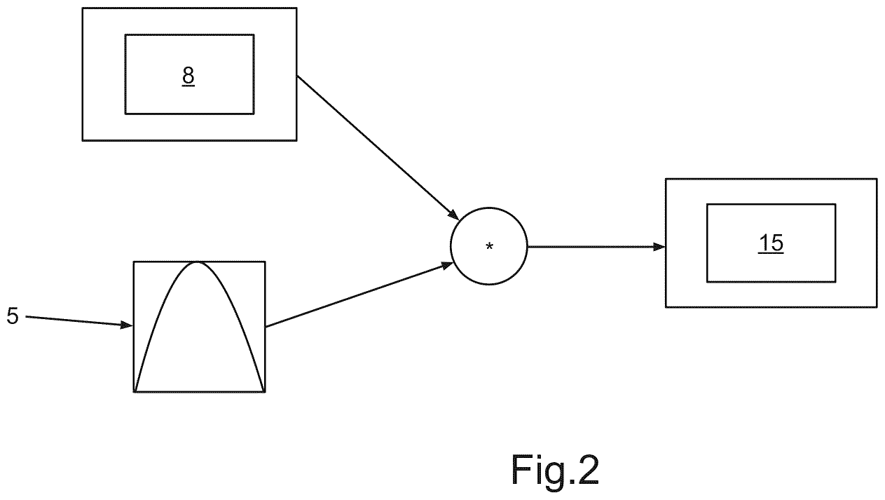

FIG. 2 shows a schematic representation of performing a deconvolution operation.

As the input values of the deconvolution operation, the control device 10 can use the processing volume 8 or the adapted processing volume 14. The control device 10 can use the laser pulse cross-sectional profile 5 as the point spread function to be applied. By applying the deconvolution operation with the laser pulse cross-sectional profile 5 as the point spread function to the processing volume 8 or the adapted processing volume 14, the deconvoluted processing volume 15 can be ascertained.

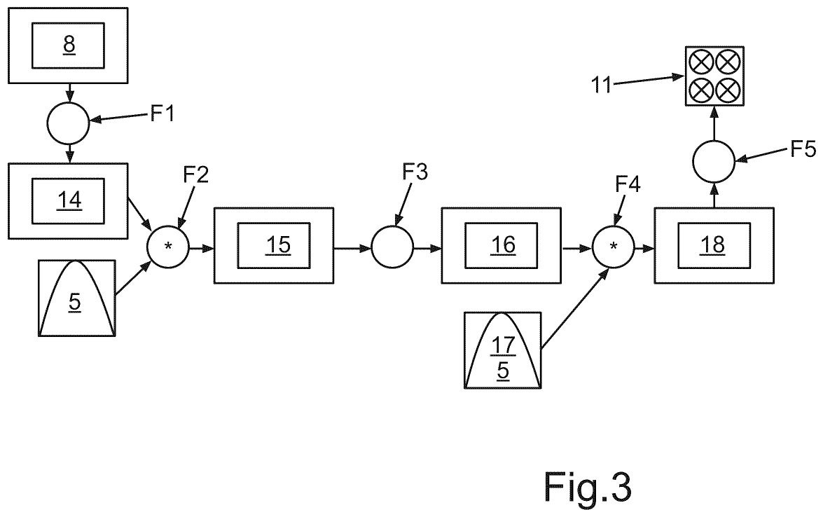

FIG. 3 shows a schematic representation of a sequence of a method for operating a processing apparatus.

The processing volume 8 can be transformed into the adapted processing volume 14 according to the adaptation method F1 by the control device 10 to compensate for varying material characteristics of the object 9 within the processing volume 8. Therein, it can be provided that the processing depth of the adapted processing volume 14 is locally changed with respect to the processing depth of the processing volume 8. For example, it can be provided that the processing depth is increased in areas of a material more difficult to ablate. Therein, the processing depth is adapted such that the fluctuations caused by material are compensated for. However, the processing depth is only adapted for the purpose of ascertaining the pulse distribution.

It can be provided that the deconvoluted processing volume 15 is ascertained by the control device 10 by means of the preset deconvolution operation F2 from the adapted processing volume 14 and the laser pulse cross-sectional profile 5. The preset deconvolution operation F2 can for example be configured to provide the deconvoluted processing volume 15 such that it can have real, integer or natural values. Physically, the values of the deconvoluted processing volume 15 can be interpreted as a number of the laser pulses 4 to be locally supplied.

The deconvoluted processing volume 15 can be transformed to a rounded deconvoluted processing volume 16 according to a predetermined rounding method F3 by the control device 10. Rounding the deconvoluted processing volume 15 can also include a subtraction of a preset value from the individual values of the deconvoluted processing volume 15. The rounding method F3 can for example be required if the deconvoluted processing volume 15 does not only include natural numbers as values and only entire laser pulses 4 can be output by the processing apparatus 1.

By means of a convolution operation F4, the rounded deconvoluted processing volume 16 can be convoluted into the rounded convoluted processing volume 18 with the laser pulse cross-sectional profile 5 as the point spread function. The rounded convoluted processing volume 18 can describe a volume, which would result upon emission of the laser pulses 4 according to the rounded deconvoluted processing volume 16. However, the rounded convoluted processing volume 18 can deviate from the preset processing volume 8 due to the rounding of the deconvoluted processing volume 15.

The deviation between the preset processing volume 8 and the rounded convoluted processing volume 18 can be ascertained by the control device 10. Depending on the deviation, a preset dithering method can be applied by the control device 10. In the dithering method, laser pulse numbers in the rounded convoluted processing volume 18 can be increased to reduce the deviation to the preset processing volume 8.

The rounded convoluted processing volume 18 can be supplied to the conversion function F5, whereby the rounded convoluted processing volume 18 can be transformed into the pulse distribution profile 11.

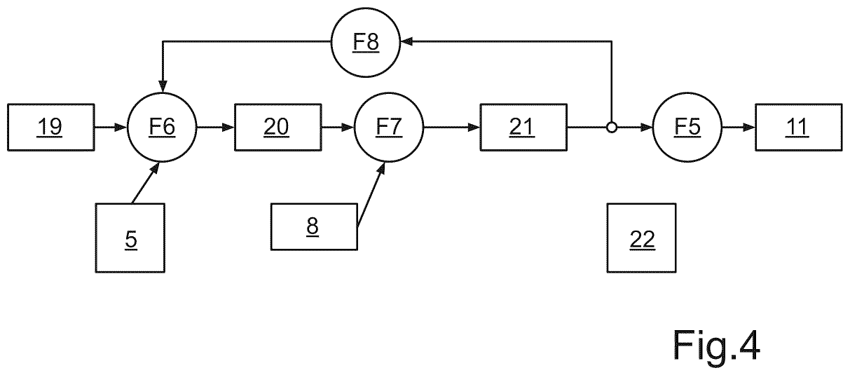

FIG. 4 shows a schematic representation of a sequence of ascertaining a pulse distribution profile according to an iterative method.

By the control device 10, an initial matrix 19 can be generated, which can include initial values, which can for example be ascertained in an estimation method for estimating an initial value for ascertaining the pulse distribution profile 11 by the control device 10. The initial matrix 19 can for example describe a first estimation for the deconvoluted processing volume 15 or the pulse distribution profile 11. By means of a convolution operation F6 with the laser pulse cross-sectional profile 5 as the point spread function, the initial matrix 19 can be transformed to a convoluted initial matrix 20 by the control device 10. The convoluted initial matrix 20 can describe a processing volume, which would result if laser pulses 3 of the laser pulse cross-sectional profile 5 would be emitted to the object 9 according to the initial matrix 19. By the control device 10, a comparison of the convoluted initial matrix 20 to the preset processing volume 8 can be effected by means of a comparison method F7. Therein, a deviation 21 between the convoluted initial matrix 20 and the preset processing volume 8 can be ascertained. The deviation 21 can be examined for satisfaction of a preset abortion criterion 22. In case of satisfaction of the abortion criterion 22, the initial matrix 19 can be transformed into the pulse distribution profile 11 by means of the conversion function F5. For example, the preset abortion criterion 22 can relate to falling below a predetermined threshold value by the deviation 21.

In case that the preset abortion criterion 22 is not satisfied, the initial matrix 19 can be adapted according to an adaptation function F8 depending on the deviation 21. Therein, at least one of the initial values of the initial matrix 19 can be changed to reduce the deviation 21. The iteration step can be repeated based on the changed initial matrix 19. The iteration method can be continued until satisfaction of the abortion criterion 22.

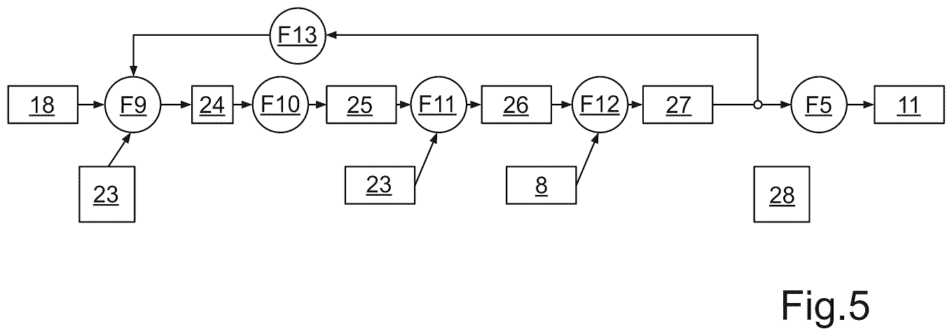

FIG. 5 shows a schematic representation of a sequence of ascertaining a pulse distribution profile according to a further iterative method.

In the iteration method, it is provided that the rounded deconvoluted processing volume 16 is convoluted into the rounded convoluted processing volume 18 by means of a convolution operation F9 corresponding to the predetermined deconvolution operation with the laser pulse cross-sectional profile 5 as the point spread function by the control device 10. The rounded, convoluted processing volume 18 can be provided as an initial value of a first step of the iteration method.

In a first iteration step of the iteration method, the rounded, convoluted processing volume 18 can be deconvoluted to a deconvoluted processing volume of the iteration step 24 by means of a deconvolution operation of the iteration step F9 with a laser pulse cross-sectional profile of the iteration step 23 as the point spread function of the iteration step and the deconvoluted processing volume of the iteration step 24 can be rounded to a rounded deconvoluted processing volume of the iteration step 25 according to the predetermined rounding method of the iteration step F10. The rounded deconvoluted processing volume of the iteration step 25 can be convoluted into a rounded convoluted processing volume of the iteration step 26 by means of a convolution operation of the iteration step F11 corresponding to the deconvolution operation of the iteration step F9 with the laser pulse cross-sectional profile of the iteration step 23 as the point spread function of the iteration step. By means of a deviation ascertaining function F12, a deviation 27 between the rounded convoluted processing volume of the iteration step 26 and the preset processing volume 8 can be ascertained. The deviation 27 can be examined for satisfaction of a preset abortion criterion 28 by the control device 10.

In case of satisfaction of the abortion criterion 28, the rounded deconvoluted processing volume of the iteration step 25 can be transformed into the pulse distribution profile 11 by means of the conversion function F5.

In case that the preset abortion criterion 28 is not satisfied, a further iteration step of the iteration method can be performed. Therein, the laser pulse cross-sectional profile of the iteration step 23, which has been used as the point spread function of the iteration step, can be adapted depending on the deviation 27 according to a predetermined adaptation function F13. The adaptation can for example include a change of a full width at half maximum of the laser pulse cross-sectional profile of the iteration step 23.

It can be provided that the iteration method is performed until the abortion criterion 28 is satisfied by the deviation 27.

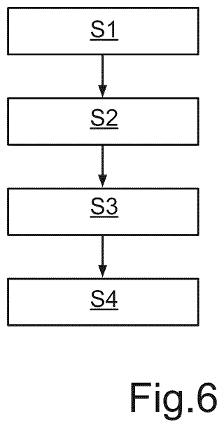

FIG. 6 shows a schematic representation of a sequence of a method for operating a processing apparatus.

The processing apparatus 1 can be configured for processing an object 9 by means of laser pulses 3 and comprise at least one laser beam source 2, which is configured for the emission of laser pulses 3, for this purpose. The processing apparatus 1 can comprise at least one beam exit device 4, which is configured to guide the respective laser pulses 3 with a predetermined laser pulse cross-sectional profile 5 according to a pulse distribution profile 11 in a processing area 7 of a preset processing volume 8 of an object 9 to be processed. The control device 10 can be configured to ascertain the pulse distribution profile 11 in the processing area 7 according to a predetermined pulse distribution ascertaining method for the preset processing volume 8 and the predetermined laser pulse cross-sectional profile 5.

It can be provided that the predetermined processing volume 8 is received by the control device 10, which can preset, which volume of which object 9 is to be removed in a first method step S1.

In a second method step S2, the control device 10 can ascertain a pulse distribution profile 11 according to a predetermined pulse distribution ascertaining method, which can preset the position of impingement of the laser pulses 3. The pulse distribution ascertaining method can provide a deconvolution operation, in which the processing volume 8 is transformed into a deconvoluted processing volume 15 with the laser pulse cross-sectional profile 5 as the point spread function according to the deconvolution operation by the control device 10.

In a third method step S3, the deconvoluted processing volume 8 can be transformed into the pulse distribution profile 11 by means of a predetermined conversion function.

In a fourth method step S4, it can be provided that the beam exit device 4 and the laser beam source 2 of the processing apparatus 1 are controlled by the control device 10 in order that the laser pulses 3 are output and guided onto the ascertained positions of impingement 6 of the pulse distribution profile 11.

Overall, the examples show, how a pulse distribution profile for removing a processing volume from an object can be provided by means of a deconvolution operation.

### LIST OF REFERENCE CHARACTERS

1 Processing apparatus
2 laser beam source
3 laser pulse
4 beam exit device
5 laser pulse cross-sectional profile
6 position of impingement
7 processing area
8 processing volume
9 object
10 control device
11 pulse distribution profile
12 computer program
13 computer-readable medium
14 adapted processing volume
15 deconvoluted processing volume
16 rounded deconvoluted processing volume
17 further laser pulse cross-sectional profile
18 convoluted rounded down processing volume
19 initial matrix
20 convoluted initial matrix
21 deviation
22 abortion criterion
23 laser pulse cross-sectional profile of the iteration step
24 deconvoluted processing volume of the iteration step
25 rounded deconvoluted processing volume of the iteration step
26 rounded convoluted processing volume of the iteration step
27 deviation
28 abortion criterion
S1-S4 method steps
F1 adaptation function
F2 deconvolution operation
F3 rounding method
F4 convolution function
F5 conversion function
F6 convolution operation
F7 comparison method
F8 adaptation function
F9 deconvolution operation of the iteration step
F10 rounding method of the iteration step
F11 convolution operation of the iteration step
F12 deviation ascertaining function
F13 adaptation function

1. A processing apparatus for processing an object by means of laser pulses, at least comprising:
at least one laser beam source, configured for emission of laser pulses,
at least one beam exit device, which is configured to guide the respective laser pulses with a predetermined laser pulse cross-sectional profile according to a pulse distribution profile in a processing area of a preset processing volume of an object to be processed, and
at least one control device, which is configured:
to ascertain the pulse distribution profile in the processing area according to a predetermined pulse distribution ascertaining method for the preset processing volume and the predetermined laser pulse cross-sectional profile , and
to adjust the ascertained pulse distribution profile in the at least one beam exit device,
to transform the preset processing volume into a deconvoluted processing volume according to a predetermined deconvolution operation
with the laser pulse cross-sectional profile as a point spread function of the deconvolution operation in the predetermined pulse distribution ascertaining method, and
to transform the deconvoluted processing volume by means of a conversion function into the pulse distribution profile.

2. The processing apparatus according to claim 1, wherein the control device is configured to adapt the preset processing volume according to a predetermined adaptation function depending on a pose of the processing area with respect to the object and/or with respect to the at least one beam exit device.

3. The processing apparatus according to claim 1, wherein the control device is configured to perform the deconvolution operation in a frequency space.

4. The processing apparatus according to claim 1, wherein the control device is configured to perform the deconvolution operation in a position space.

5. The processing apparatus according to claim 1, wherein the control device is configured to perform a non-negative deconvolution as the deconvolution operation.

6. The processing apparatus according to claim 1, wherein the control device is configured to perform an integer deconvolution as the deconvolution operation.

7. The processing apparatus according to claim 1, wherein the control device is configured to perform a binary deconvolution as the deconvolution operation.

8. The processing apparatus according to claim 1, wherein the control device is configured to convert the deconvoluted processing volume to a rounded deconvoluted processing volume by means of a rounding method.

9. The processing apparatus according to claim 1, wherein the control device is configured:
to convert the deconvoluted processing volume to a rounded deconvoluted processing volume by means of a rounding method,
to assign a value of 0 in the rounding method to values of the deconvoluted processing volume with decimal places between 0 and 0.3 exclusive in the rounded deconvoluted processing volume,
to assign a laser pulse with a predetermined lower energy to values of the deconvoluted processing volume between 0.3 inclusive and 0.7 inclusive, and
to assign an entire laser pulse to values of the deconvoluted processing volume from 0.7 exclusive in the rounded deconvoluted processing volume.

10. The processing apparatus according to claim 1, wherein the control device is configured:
to convert the deconvoluted processing volume to a rounded deconvoluted processing volume by means of a rounding method, and
to apply a post-hoc dithering method to the rounded deconvoluted processing volume depending on a deviation of the rounded deconvoluted processing volume from the deconvoluted processing volume for reducing the deviation.

11. The processing apparatus according to claim 1, wherein the control device is configured to convert the deconvoluted processing volume to a rounded down deconvoluted processing volume by means of a rounding method, wherein the control device, in a refining method, is configured:
to convolute the rounded down deconvoluted processing volume into the convoluted rounded processing volume by means of a predetermined convolution operation with the laser pulse cross-sectional profile as a point spread function,
to deconvolute the convoluted rounded processing volume into a further deconvoluted processing volume according to a predetermined further deconvolution operation with a further laser pulse cross-sectional profile as a further point spread function of the deconvolution operation, and
to transform the further deconvoluted processing volume into the pulse distribution profile.

12. The processing apparatus according to claim 1, wherein the control device is configured:
to convert the deconvoluted processing volume to a rounded deconvoluted processing volume by means of a rounding method, and wherein the control device is configured for
to perform an iterative adaptation method, wherein the control device is configured, in the iteration method, in which the control device is configured:
to convolute the rounded deconvoluted processing volume into a rounded convoluted processing volume by means of a convolution operation corresponding to the predetermined deconvolution operation with the laser pulse cross-sectional profile as the point spread function,
in a respective step of the iteration iterative adaptation method, to deconvolute the rounded convoluted processing volume to a deconvoluted processing volume of an iteration step with a deconvolution operation of the iteration step with a laser pulse cross-sectional profile of the iteration step as the point spread function of the iteration step,
to round the deconvoluted processing volume of the iteration step to a rounded deconvoluted processing volume of the iteration step according to a predetermined rounding method,
to convolute the rounded deconvoluted processing volume of the iteration step into a rounded convoluted processing volume of the iteration step by means of a convolution operation of the iteration step corresponding to the deconvolution operation of the iteration step with the laser pulse cross-sectional profile of the iteration step as the point spread function of the iteration step,
to ascertain a deviation between the rounded convoluted processing volume of the iteration step and the preset processing volume,
to examine the deviation for satisfaction of a preset abortion criterion and to transform the rounded deconvoluted processing volume into the pulse distribution profile in case of satisfaction of the abortion criterion, and
in case that the preset abortion criterion is not satisfied, to perform a further iteration step of the iterative adaptation method, wherein the laser pulse cross-sectional profile of the further iteration step is adapted as the point spread function of the further iteration step depending on the deviation according to a predetermined adaptation function and/or the deconvolution operation as well as the convolution operation of the iteration step are adapted depending on the deviation.

13. The processing apparatus according to claim 1, wherein the control device is configured:
to convert the deconvoluted processing volume to a rounded deconvoluted processing volume by means of a rounding method, and
to perform an iterative adaptation method, in which the control device is configured:
to ascertain an initial matrix,
in a respective step of the iterative adaptation method, to convolute the initial matrix by means of a convolution operation with the laser pulse cross-sectional profile as the point spread function into a convoluted initial matrix,
to compare the convoluted initial matrix to the preset processing volume, and to ascertain a deviation between the convoluted initial matrix and the preset processing volume,
to examine the deviation for satisfaction of a preset abortion criterion,
in case of satisfaction of the abortion criterion, to transform the initial matrix into the pulse distribution profile by means of the conversion function, and
in case that the preset abortion criterion is not satisfied, to perform a further iteration step of the iterative adaptation method, wherein the initial matrix is adapted according to an adaptation function depending on the deviation.

14. A method for operating a processing apparatus for processing an object by means of laser pulses, comprising the following steps performed by at least one control device:
ascertaining a pulse distribution profile in a processing area according to a predetermined pulse distribution ascertaining method for a preset processing volume of an object to be processed, and a predetermined laser pulse cross-sectional profile, comprising the partial steps:
transforming the preset processing volume into a deconvoluted processing volume according to a predetermined deconvolution operation with the laser pulse cross-sectional profile as a point spread function of the deconvolution operation,
transforming the deconvoluted processing volume into the pulse distribution profile by means of a predetermined conversion function,
adjusting the ascertained pulse distribution profile in at least one beam exit device to guide respective laser pulses with a predetermined laser pulse cross-sectional profile according to the pulse distribution profile into the processing area of the preset processing volume of the object to be processed, and
controlling at least one laser beam source for emission of the laser pulses.

15. A control device configured for controlling a processing apparatus according to the method of claim 14.

16. (canceled)

17. A computer-readable medium for storing a computer program, the computer program comprising commands which cause a processing apparatus to execute the method according to claim 14.
